(19) United States
(12) Patent Application Publication
(10) Pub. No.: US 2017/0265494 A1
(43) Pub. Date: Sep. 21, 2017

(54) CHEWING GUM INCLUDING NANOZOME ENCAPSULATED CANNABIDIOL

(71) Applicant: International Consolidated Companies, Inc., Aventura, FL (US)

(72) Inventor: Antonio Uccello, III, Aspen, CO (US)

(73) Assignee: International Consolidated Companies, Inc., Aventure, FL (US)

(21) Appl. No.: 15/614,362

(22) Filed: Jun. 5, 2017

(51) Int. Cl. A23G 4/20; A61K 47/18; A61K 9/68; A61K 47/24; A23G 3/54; A61K 9/20

(52) U.S. Cl. CPC A23G 4/205; A23G 3/545; A61K 9/2018; A61K 9/0058; A61K 47/24; A61K 47/18; A61K 31/685

(57) ABSTRACT

Chewing gum including a liquid center, a chewy layer surrounding the liquid center, and a coating surrounding the chewy layer. The liquid center includes a nanozome encapsulated dose of a cannabinoid such as cannabidiol of approximately 1-30 mg. The nanozome is preferably a phospholipid such as a Phosphatidyl Choline. In another embodiment the nanozome is unsaturated Phosphatidyl Choline to improve bioavailability of the cannabidiol, or other cannabinoid.

### FIELD OF THE INVENTION

This invention pertains to nanozome encapsulated cannabinoids, and in particular to chewing gum products including the same.

### BACKGROUND AND SUMMARY OF THE INVENTION

In the 1980's, a number of gum products entered the U.S. Market. Some of these gum products included a liquid center. An example is the Freshen Up® brand gum product marketed as chewing gum with liquid center that would burst when chewed. The center was colored and flavored to garner appeal with consumers.

A product called Rescue® brand gum marketed by Bach Flower Remedies Limited is currently sold to provide homeopathic medicine in a liquid center. The chewing gum includes various herbs in the liquid center. Each piece is made from a thickener such as gum arabic, natural flavors, various acids, and a humectant including glycerol. The surface has a glaze of carnuba wax and talc. It is sweetened with isomalt and the following sugar alcohols: sorbitol, maltitol, amnnitol and xylitol.

While this homeopathic delivery mechanism is an improvement over the tiny homeopathic pills or tinctures that are usually sold, there is no mechanism known to the this inventor in the formulation of this brand of gum that readily improves bioavailability over other modes of oral delivery.

International Patent Publication No. WO 2001003668 A, published Jan. 18, 2001 to Applicant, Her Majesty The Queen In Right Of Canada as represented by the Minister of National Defence of Her Majesty's Canadian Government, et al., discloses a liposomal composition including Cannabidiol to increase the efficacy the Cannabidiol. Nano-scale phospholipids including phosphatidylcholine are disclosed. Encapsulation of cannabinoids with nano-scale lipids improves the efficacy of cannabinoids via pulmonary administration.

While this publication represents a step forward in the art of pulmonary administration of cannabinoids, recent consumer demand indicates that alternate ways to ingest cannabinoids are desired.

Phosphatidyl Choline (PC) is an abundant phospholipid in the cell walls of numerous organs of the human body. The skin, liver, and brain all require sufficient PC to function properly. The cell walls are semi-permeable to control the movement of substances in and out of the cell. Sufficient PC is critical to maintain balanced permeability of the cell membrane.

Phosphatidyl Choline is suitable for encapsulating nutraceutical molecules, including Cannabidiol. Phosphatidyl Choline can be extracted from plant-sourced lecithin, including soy lecithin and lecithin from sunflowers. When nano-scale sized phospholipids are used to encapsulate nutraceutical ingredients, improved bio-availability is often achieved.

Nanozomes is a term that refers to liposomes in the nanometer range. This nanometer range is preferred for bio-availability of nutraceuticals and pharmaceuticals when administered orally.

### BRIEF DESCRIPTION OF THE DRAWING

FIG. 1 is a perspective cross-sectional view of a piece of chewing gum in accordance with the present invention.

### DETAILED DESCRIPTION

Chewing gum according to the present invention includes a liquid center including a nanozome encapsulated cannabinoid, a chewy layer surrounding the liquid center and a coating including a sweetener surrounding the chewy layer. Cannabinoids include any phytocannabinoids derived from Cannabis Sativa, or a synthesized cannabinoid, or an endocannabinoid. The cannabinoids are preferably isolated in a purified form having a 90-99.9% purity. Terpenes derived from Cannabis Sativa having synergistic effects may be added in desired ratios. Cannabinoids are generally defined as any compound or molecule that directly effects the cannabinoid receptors CB1 or CB2 in humans.

Preferably, the cannabinoid utilized is a purified form of Cannabidiol (CBD), Tetrahydracannabinol (THC), Cannnabinol (CBN), Cannabigerol (CBG), or combinations thereof. An exemplary list of cannabinoids follows:

CBG (Cannabigerol)

CBC (Cannabichromene)

CBL (Cannabicyclol)

CBV (Cannabivarin)

THCV (Tetrahvdrocannabivarin)

CBDV (Cannabidivarin)

CBCV (Cannabichromevarin)

CBGV (Cannabigerovarin)

CBGM (Cannabigerol Monomethyl Ether)

THC (Tetrahydrocannabinol)

THCA (Tetrahydrocannbinolic acid)

CBD (Cannabidiol)

CBDA (Cannabidiolic Acid)

The chewy layer and coating are formulated in accordance with known ways of manufacture of chewing gum. In one embodiment the coating is a hardened and sweetened shell that resists heat and impact to maintain product integrity until consumed. The shell may be sweetened with a sucrose-based formulation, or a sugar-free formulation utilizing a polyol i.e. a sugar alcohol, or a polyol mixed with a high intensity sweetener such as a steviol glycoside.

In one embodiment of the invention, the nanozome is a phospholipid such as a Phosphatidyl Choline. In another embodiment the phospholipid is unsaturated, such as unsaturated Phosphatidyl Choline.

Preferably, the phospholipid is derived from a vegetable-sourced lecithin such as soy lecithin or sunflower lecithin. In another embodiment, the phospholipid is derived from non-plant sources such as egg yolk.

In other embodiments, the nanozome is a glycerophospholipids selected from the group consisting of: phosphatidylcholine, phosphatidylethanolamine, phosphatidylinositol, phosphatidylserine, and phosphatidic acid. These phospholipids can be used to encapsulated Cannabidiol to improve bioavailability, latency in vivo, and water solubility to optimize absorption through the mucusol membranes of the upper digestive tract, including the mouth, and to improve bioavailability on a cellular level.

The nanozome can be adapted in size and structure to optimize absorption into particular organs of the body, ie. musculature, nervous system, skeletal system, etc. Typically the nanozome has a size within the range of 50-800 nanometers.

There are various ways to extract glycerophospholipid from a source material. In one embodiment, the glycerophospholipid is extracted chemically using a non-polar solvent. In another embodiment, the glycerophospholipid is extracted mechanically, or mechanically with heat. In a variation of the invention, the glycerophospholipid is extracted mechanically by water degumming the extracted oil of seeds, where the extracted oil is cold pressed.

In a preferred embodiment of the invention, the glycerophosphoilipid is extracted from sunflower seed oil to be hypoallergenic. This extraction is performed by mechanical means.

In the liquid center of the chewing gum according to one aspect of the invention, the glycerophospholipid includes phosphatidylcholine in a concentration of at least 19%. The sweetener includes a steviol glycoside. Preferably, the steviol glycoside is Rebaudioside A that has been subject to enzymatic glycosylation to minimize flavor defects.

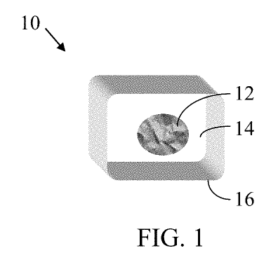

FIG. 1 shows a piece of chewing gum in accordance with the present invention. The chewing gum is generally designated with the reference numeral 10. The chewing gum 10 has a center 12. The center 12 includes liposomal encapsulated cannabinoids including, in one embodiment of the invention, Cannabidiol. Preferably the center is liquid, but the center could also be formulated into a gel or other non-liquid form. It can be appreciated that any cannabinoid or combination thereof can be utilized as set forth herein.

A chewy layer 14 surrounds the liquid center. The chewy layer includes gum arabic and flavorings. A coating 16 surrounds the chewy layer. The coating is heat resistant to protect the piece of gum in ambient conditions including sunlight and ambient heat.

I claim:

1. Chewing gum comprising:
a liquid center including a nanozome encapsulated cannabinoid;
a chewy layer surrounding the liquid center; and
a coating including a sweetener surrounding the chewy layer.

2. The chewing gum as set forth in claim 1, wherein the nanozome is a phospholipid.

3. The chewing gum as set forth in claim 2, wherein the phospholipid is Phosphatidyl Choline.

4. The chewing gum as set forth in claim 3, wherein the phospholipid is unsaturated.

5. The chewing gum as set forth in claim 4, wherein the phospholipid is derived from lecithin.

6. The chewing gum as set forth in claim 5, wherein the phospholipid is derived from soy lecithin.

7. The chewing gum as set forth in claim 4, wherein the phospholipid is derived from sunflower lecithin.

8. The chewing gum as set forth in claim 4, wherein the phospholipid is derived from egg yolk.

9. The chewing gum as set forth in claim 1, wherein the nanozome is a glycerophospholipids selected from the group consisting of:
phosphatidylcholine,
phosphatidylethanolamine, phosphatidylinositol, phosphatidylserine, and phosphatidic acid.

10. The chewing gum as set forth in claim 9, wherein the glycerophospholipid is extracted chemically using a non-polar solvent.

11. The chewing gum as set forth in claim 9, wherein the glycerophospholipid is extracted mechanically.

12. The chewing gum as set forth in claim 9, wherein the glycerophospholipid is extracted mechanically by water degumming the extracted oil of seeds, where the extracted oil is cold pressed.

13. The chewing gum as set forth in claim 9, wherein the glycerophospholipid is extracted mechanically and with heat.

14. The chewing gum as set forth in claim 9, wherein the glycerophosphoilipid is extracted from sunflower seed oil to be hypoallergenic.

15. The chewing gum as set forth in claim 9, wherein the glycerophosphoilipid is extracted from sunflower seed oil by mechanical means.

16. The chewing gum as set forth in claim 14, wherein the glycerophospholipid includes phosphatidylcholine in a concentration of at least 19%.

17. The chewing gum as set forth in claim 16, wherein the sweetener includes a steviol glycoside.

18. The chewing gum as set forth in claim 16, wherein the steviol glycoside is Rebaudioside A.

19. The chewing gum as set forth in claim 18, wherein the Rebaudioside A has been subject to enzymatic glycosylation to minimize flavor defects.
